(19) United States
(12) Patent Application Publication
(10) Pub. No.: US 2005/0129630 A1
(43) Pub. Date: Jun. 16, 2005

(54) Utilization of natural pigments from lichens, cyanobacteria, fungi and plants for sun protection

(76) Inventors: Enk David Claes, Baka (IL); Morris Srebnik, Mevasseret Zion (IL); Ovadia Lev, Yefeh Nof (IL); Malka Hochberg, Beth Horon (IL); Inka Dor, Ein Karem (IL); Avital Torres-Kerner, Haifa (IL); Valery M Dembitsky, Jerusalem (IL)

Correspondence Address: BROWDY AND NEIMARK, P.L.L.C.;624 NINTH STREET, NW, SUITE 300, WASHINGTON, DC, 20001-5303, US

(21) Appl. No.: 10/488,426

(22) PCT Filed: Sep. 3, 2002
(86) PCT No.: PCT/IL02/00725

(30) Foreign Application Priority Data
Sep. 3, 2001 (IL) 145250

(51) Int. Cl. A61K 7/42; A61K 31/7052; C07H 17/02

(52) U.S. Cl. 424/59; 514/23; 536/17.4

(57) ABSTRACT

The present invention relates to a natural extract from fungus, cyanobactria, plants, lichens or a mixture thereof having an ultra violet absorbency in the range of 220 nm to 425 nm wherein said extract is obtained by contacting said fungus, cyanobacteria, plant, lichen or a mixture thereof with an C₁₋₇-alcoholic solution. The present invention further relates to a compound of formula I purified from fungus and to cosmetical compositions comprising a natural extract or a compound of formula I.

### FIELD OF THE INVENTION

This invention relates to natural extracts, novel compounds obtained therefrom and their use for medical and cosmetic sun-protection agents.

### BACKGROUND OF THE INVENTION

Exposure to ultraviolet radiation (UVR) from the sun plays a causal role in acute and chronic skin damage such as sunburn, skin cancer, immunosuppression, and photoaging of the skin. These consequences of sun exposure are attracting considerable attention due to an alarming increase in the incidence of sun-related skin cancers. Major culprits of increased sun-related morbidity include changes in life style with more time spent in outdoor recreational activities resulting in significant augmentation in the amount of UVR received and depletion of stratospheric ozone, which is the Earth's protection layer against hazardous radiation. To amend for these dangerous developments, a sun avoidance strategy has been advocated in which the topical application of sunscreens constitutes a cornerstone. However, the increased use of sunscreens raises several concerns: Most sunscreens do not effectively filter out all the detrimental wavelengths of sun light. Second, even though sunscreens prevent sunburn, little is known regarding the threshold or dose-response for UVR-induced effects on other endpoints such as immune suppression and DNA damage. Finally, there is increasing body of evidence that presently used topical sunscreens might undergo UV-induced photooxidation and form potentially toxic metabolites.

Commercial sunscreen formulations make use of both organic and inorganic agents as the components of the formulation. Organic sunscreens have been the mainstay of sunscreen formulation for decades. They are classified as derivatives of anthranilates, benzophenones, camphors, cinnamates, dibenzoylmethanes, p-aminobenzoates or salicylates. These aromatic compounds absorb a specific portion of the UVR spectrum that is generally re-emitted at a less energetic, longer wavelength, or used in a photochemical reaction. The organic sunscreens are almost always used in combination in order to provide a high sun protection factor (SPF) and to broaden the absorption spectrum. Inorganic sunscreens, containing either zinc oxide or titanium dioxide, are becoming increasingly popular, mainly due to their ability to filter longer UVA wavelengths. The inorganic particles block radiation by scattering and absorbing incident photons.

Search for a new generation of sunscreens stems from the various drawbacks the present sunscreen agents possess, including photo- and nonphotoinduced skin sensitivity and photogenotoxicity. Naturally occurring UV filters in the form of pigments are abundant and might constitute attractive candidates for new effective and nontoxic sunscreens. In addition to melanin and flavonoides, they include scytonemins found in cyanobacteria with a recently elucidated structure (Proteau et al (1993) Experimentia 49:825-829). This pigment, the first shown to be an effective photostable UV shield in prokaryotes, is a dimeric molecule of indole and phenol subunits. The scytonemin absorbs strongly and broadly in the spectral region of 325-425 nm (UVA) but also has an absorption in the UVB (280-320 nm) and UVC (<250 nm) regions (U.S. Pat. No. 5,461,070). Mycosporine is another family of water-soluble, ultra violet-absorbing metabolites found in cyanobacteria with an UV absorption peak in the UVB range. The elucidated structure of mycosporine is cyclohexenone chromophore conjugated with the nitrogen of an amino acid or an amino alcohol. A variety of specific mycosporin amino acids were identified and their distribution in various groups has been described (Karentz et al. (1991) Marine Biology 108, 157-166).

### SUMMARY OF THE INVENTION

The present invention is based on the fact that extracts from various natural sources and novel compounds obtained therefrom and their derivatives, absorb efficiently ultra violet radiation and thus may be used as effective sun-protection agents.

Thus the present invention relates to novel natural extracts isolated from fungus, cyanobacteria, plants, lichens or a mixture thereof having ultra violet absorbency in the range between 220 nm and 425 nm. The extraction is done by contacting said fungus, cyanobacteria, plants, lichens or a mixture thereof with a C₁₋₇-alcoholic solution. The alcoholic solution may be an aqueous solution or comprise a hydrophobic organic solvent. The fungus is Collema associated fungus, the plant is chosen from the group comprising of Pecan nut, Cichorium endivia, Eriobotrya nut or mixtures thereof, the lichen is Xanthoria and the cyanobacteria is chosen from the group consisting of Spirulina or Aphanizomenon or mixture is thereof.

The invention further relates to a compound extracted and isolated from Collema lichens and its derivatives of formula (I): 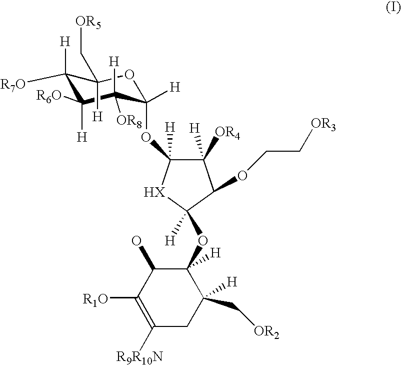
wherein R¹-R⁸ which may be the same or different are selected from the group comprising of hydrogen, a C₁-C₁₀-alkyl or acyl group; R⁹ and R¹⁰ which may be the same or different are selected from the group comprising of C₁-C₁₀-alkyl, aryl, hydrogen or an acyl group; and X is NR, oxygen or sulfur, wherein R is hydrogen, alkyl or aryl.

The invention further relates to cosmetic formulations providing protection for skin from the hazardous effects of ultra violet (UVA and UVB) irradiation, comprising an effective amount of an alcoholic extract from fungus, cyanobacteria, plant, lichen, a mixture thereof or an effective amount of a compound of formula (I). Such cosmetic formulations may further comprise at least one additional sun-protecting agent.

The invention still further relates to the use of an effective amount of the extract of the present invention or of a compound of formula I, optionally together with at least one additional sun-protecting agent for the preparation of a sunscreen formulation providing protection from ultra violet (UVA and UVB) irradiation.

### BRIEF DESCRIPTION OF THE DRAWINGS

In order to understand the invention and to see how it may be carried out in practice, a preferred embodiment will now be described, by way of non-limiting example only, with reference to the accompanying drawings, in which:

FIGS. 1A and 1B show the ultra violet spectrum of extracts from the fungus Aphanizomenon flos aquae (1A) and Collema associated fungus (CAF) (1B).

FIGS. 2A and 2B show the ultra violet spectrum of extracts from Pecan nut (2A) and from Cichorium endivia subsp. Divaricatum (2B).

FIGS. 3A, 3B and 3C show the ultra violet spectrum of extracts from Eriobotrya nuts (3A), from Spirulina (3B) and from Xanthoria (3C).

FIG. 4 shows results from in vivo testing of the efficacy of protection of skin of an arm exposed to U.V. irradiation when covered with lotion comprising of (1) Collema associated fungus; (2) Cichorium; (3) Eriobotrya; (4) Pecan; and (5) olive oil (serving as control).

FIGS. 5A and 5B show the ultra violet spectrum in the range of 290 nm to 400 nm taken in increments of 5 nm of extracts Spirulina (5A) and from Aphanizomenon flos aquae (5B) showing the UVA/UVB ratio.

FIGS. 6A and 6B show the ultra violet spectrum in the range of 290 nm to 400 nm taken in increments of 5 nm of two comparative samples “nivea 3 star” (6A) and “boots soltan 4 star” showing the UVA/UVB ratio.

FIG. 7 shows the ultra violet spectrum of the purified compound (compound I) isolated from the extract of Collema lichens.

FIG. 8 is a I-Dimensional NMR spectrum of the purified compound I together with its chemical formula.

FIGS. 9A and 9B show the Mass Spectrum of the purified compound I. FIG. 9A shows the Time of Flight Electron Spray Ionization (TOF ESI). FIG. 9B is a simulation of the proposed molecular peak at 497.026 corresponding to C₁₉H₃₂N₂O₁₃ (MH).

FIGS. 10A and 10B show the in vitro ultra violet (UVB) protection obtained by the compound of formula I in terms of survival of cultured human keratinocytes (10A) and the protection in terms of pyrimidine dimer formation in cultured human keratinocytes (10B).

FIGS. 11A_{(I,II,III)}-D_{(I,II,III)} show the in vitro ultra violet protection in terms of survival of cultured human keratinocytes (I), the protection in terms of immunosuppression as measured by expression of IL-6 mRNA (II) and the protection in terms of pyrimidine dimer formation in cultured human keratinocytes (III) after ultraviolet (UVB) radiation. Protection was measured with the natural extracts of: CAF (11A), Cichorium (11B), Pecan (11C) and Eriobotrya (11D).

### DETAILED DESCRIPTION OF THE INVENTION

The invention will now be described with reference to some non-limiting specific embodiments. The invention will first be illustrated in reference to the attached drawings to be followed by a more detailed description below.

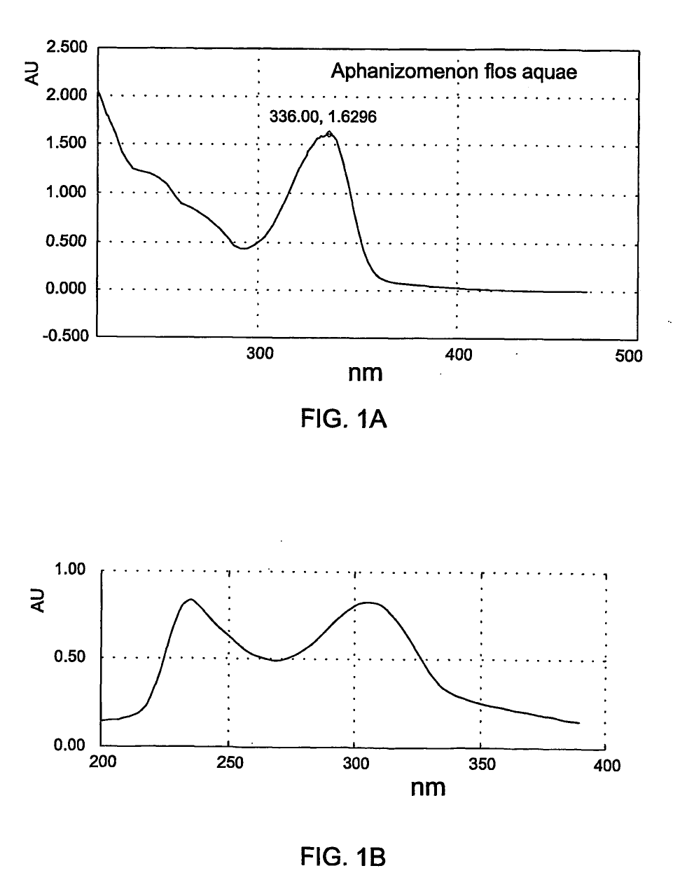

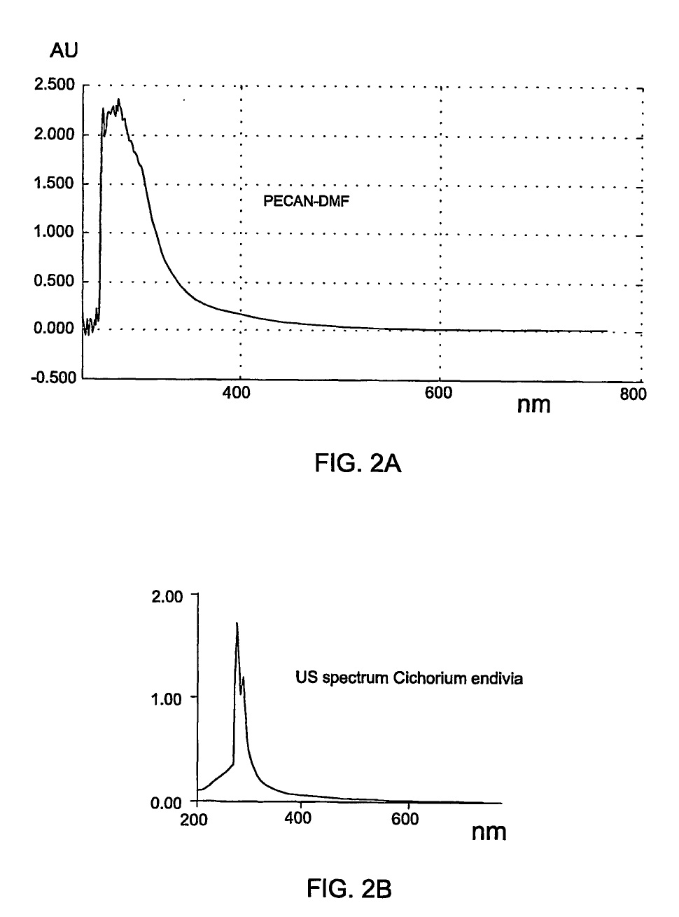

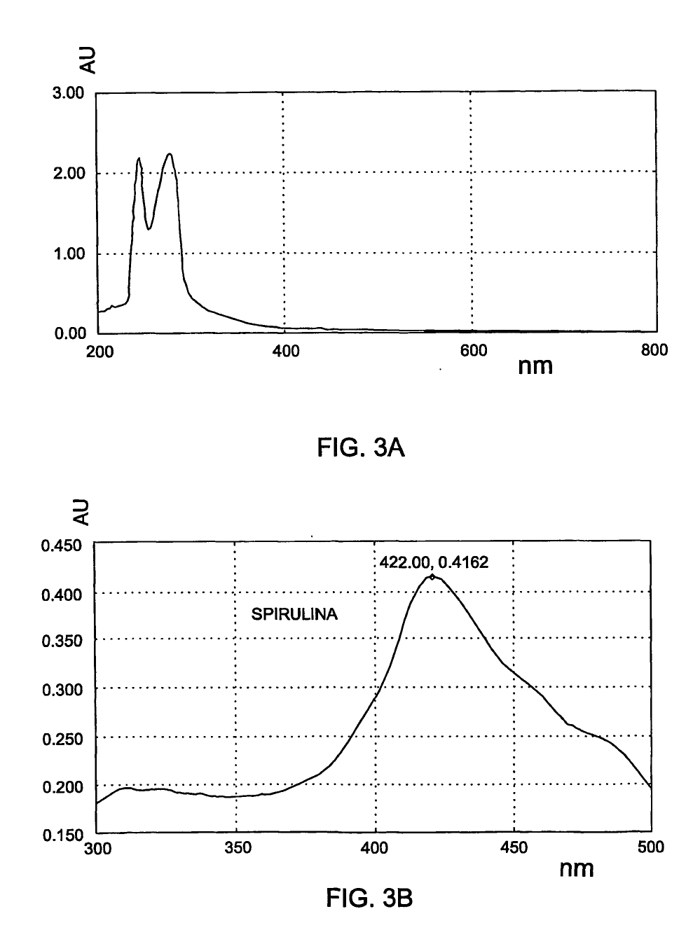

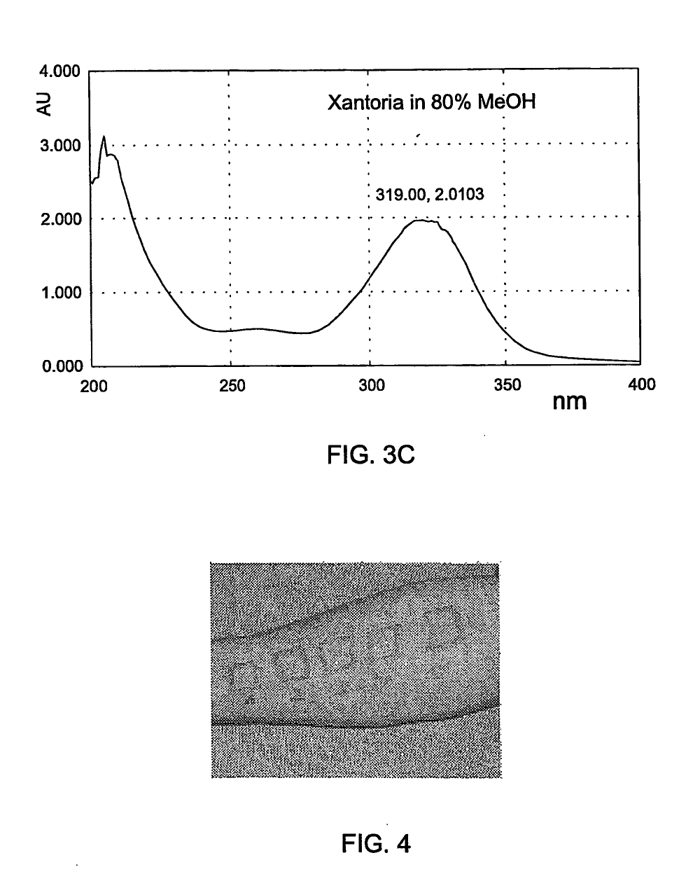

FIGS. 1A and 1B show the ultra violet spectrum of the extracts obtained from the cyanobacteria Aphanizomenon flos aquae and from the fungal part of the Collema, hereinafter termed Collema associated fungus (CAF). These two extracts posses a strong absorption in the ultra violet region, the former at 336 nm and the latter at 250 nm and 311 nm. FIGS. 2A and 2B show the ultra violet spectrum of the extracts obtained from the plants pecan and Cichorium endivia showing an ultra violet absorption at 306 nm and 283 nm, respectively. FIGS. 3A, 3B and 3C show the ultra violet spectrum of the extracts obtained from Eriobotrya nuts, from the cyanobacteria Spirulina and from lichen Xanthoria showing ultra violet absorption at 306 nm, 422 nm and 319 nm, respectively. FIG. 4 shows the in vivo efficacy of protection of the skin in the volar forearm exposed to ultra violet irradiation after the arm is covered with a lotion comprising the alcoholic extracts from (1) CAF (2) Cichorium, (3) Eriobotrya (4) and Pecan, all dissolved in olive oil and (5) olive oil taken as a control. It is clearly shown that while the area covered with olive oil is turned red upon the ultra violet exposure, the four confined areas where the skin was covered with lotions comprising one of the extracts of the present invention, the skin is unharmed. Such a comparison clearly demonstrates the sun-protecting effect of the extracts.

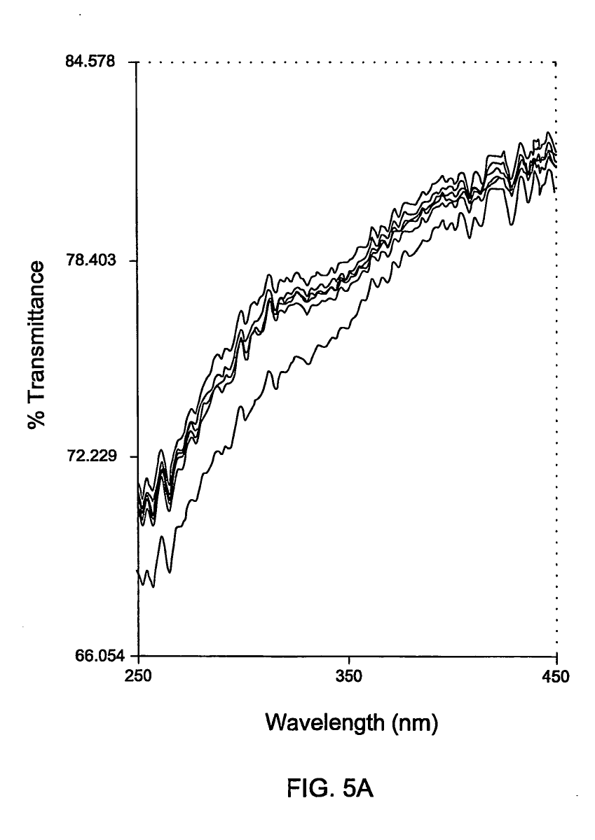

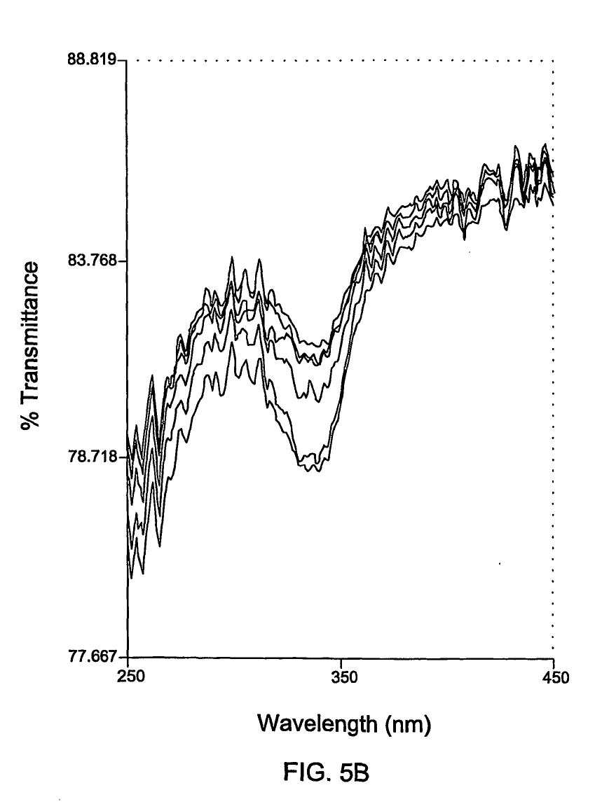

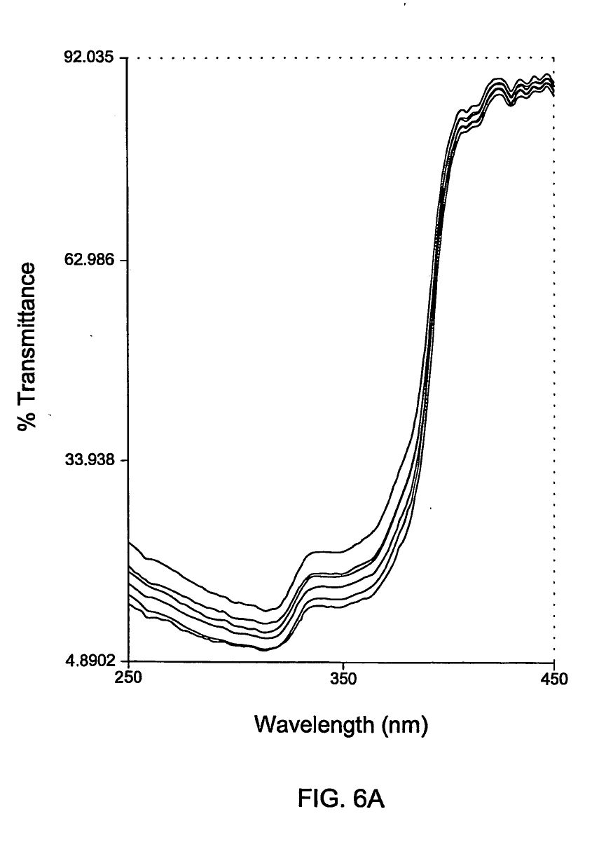

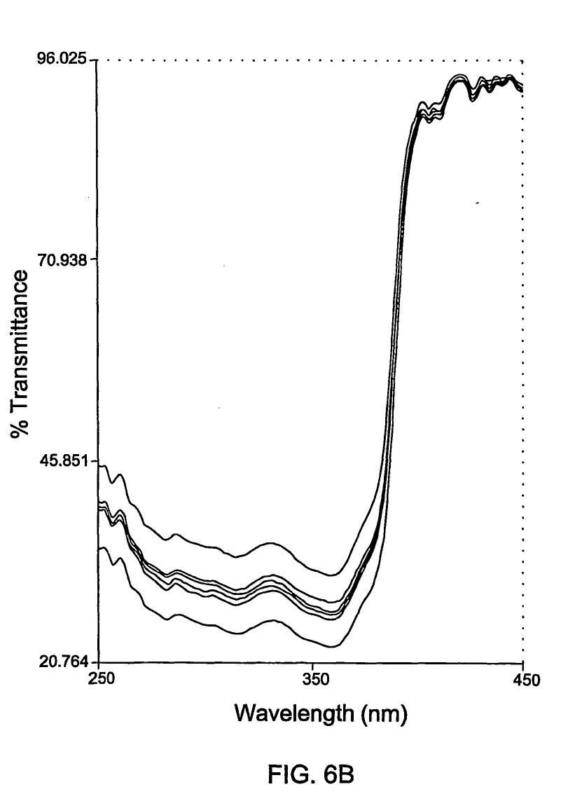

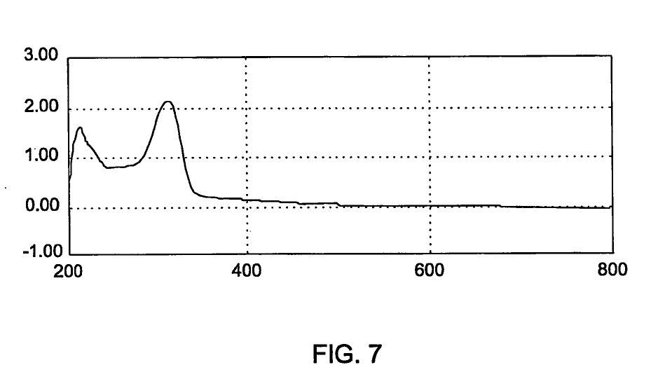

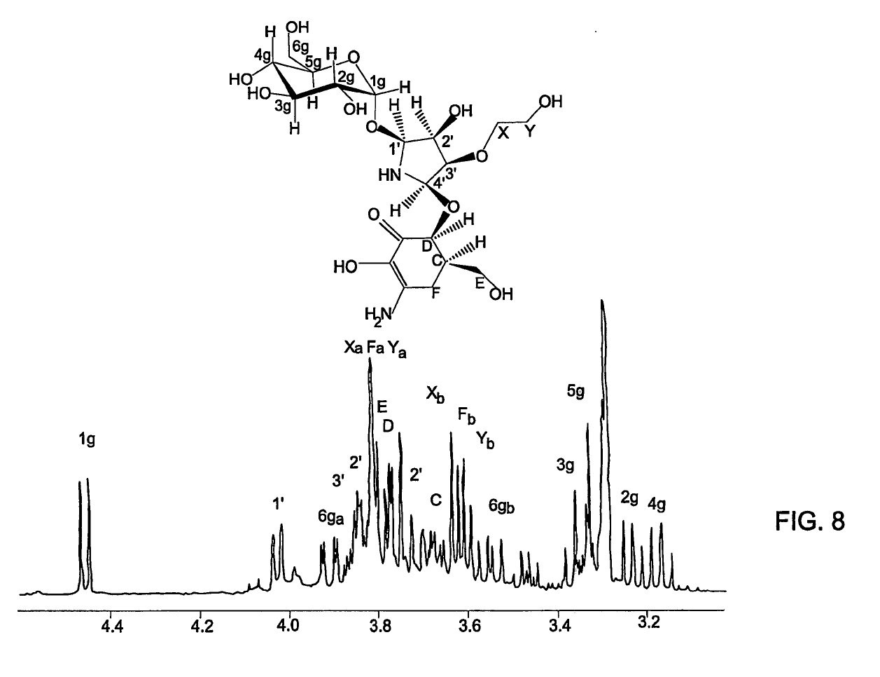

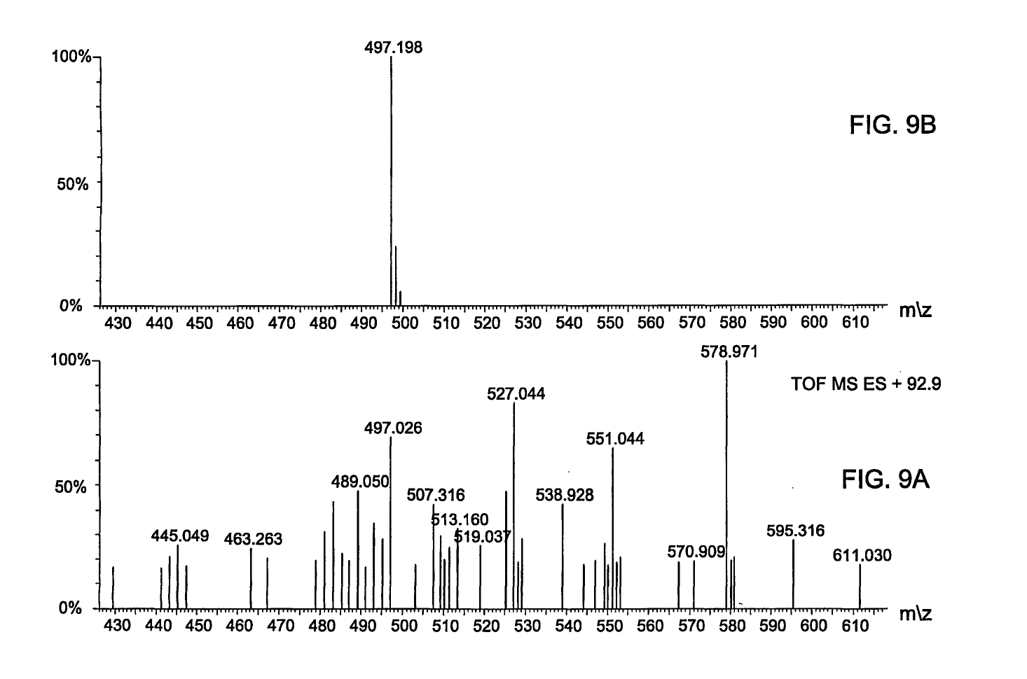

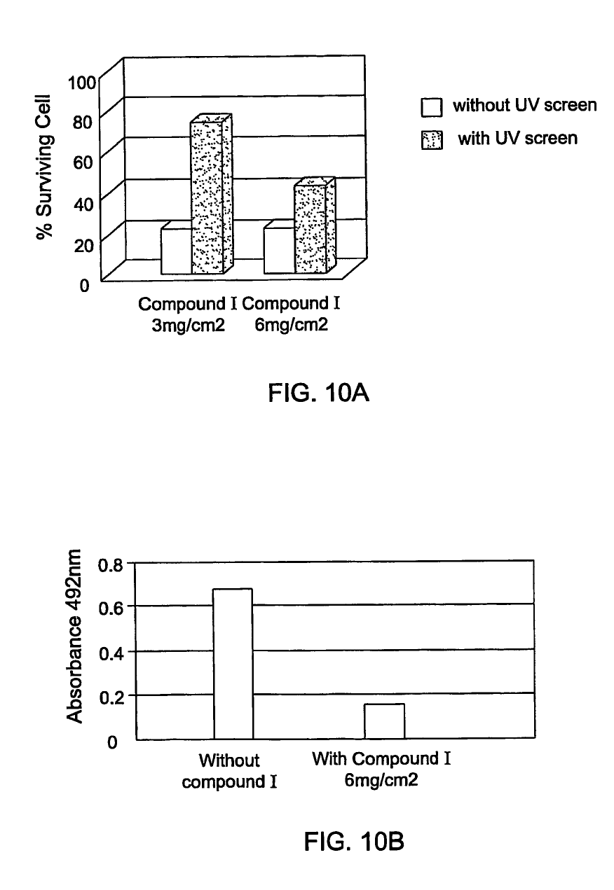

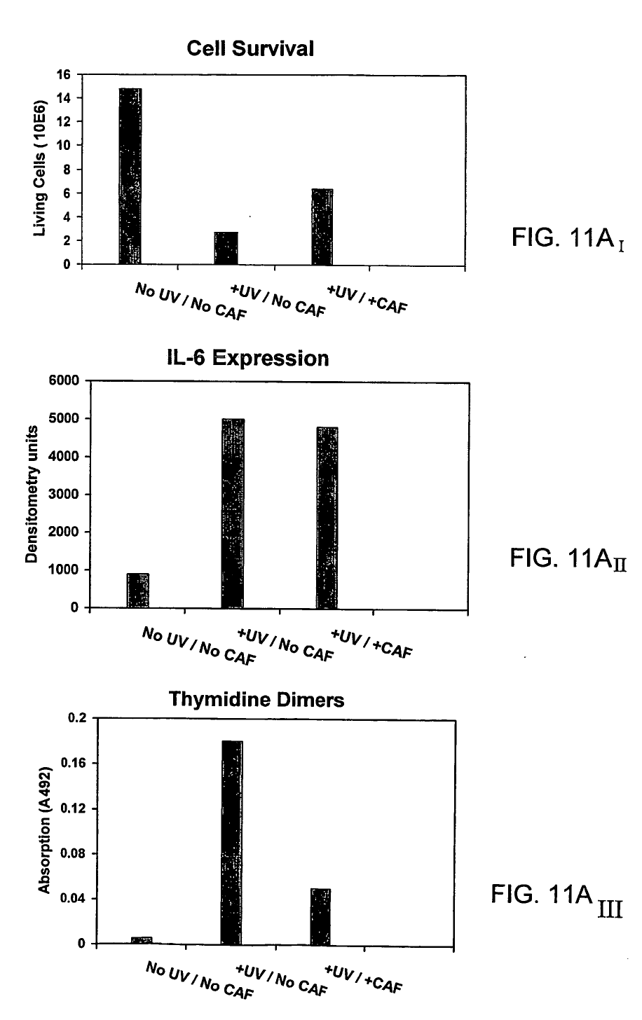

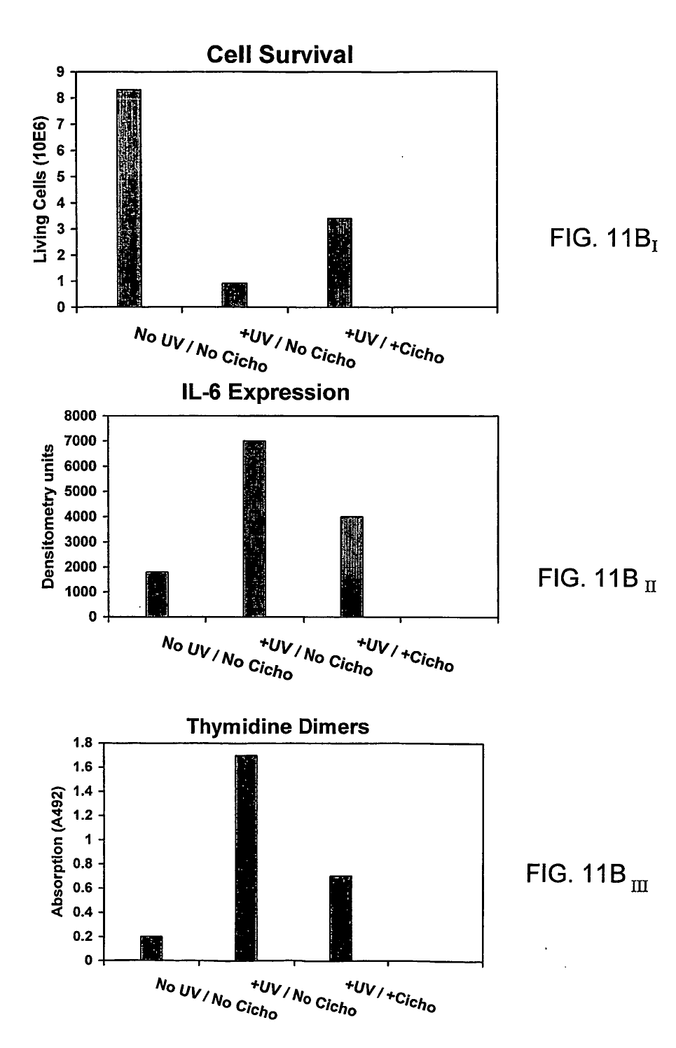

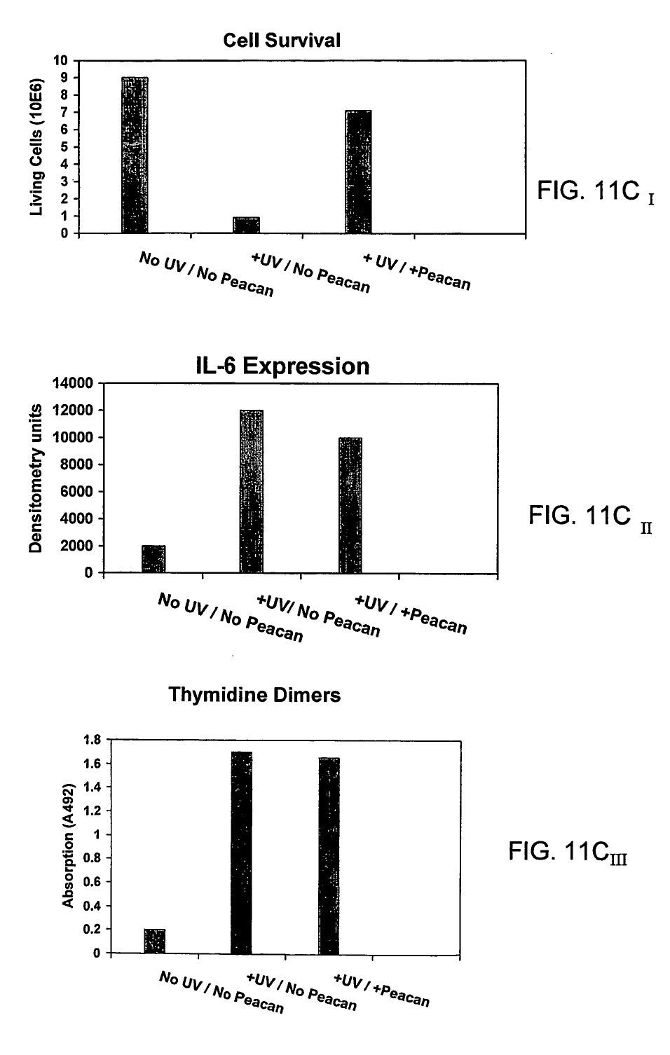

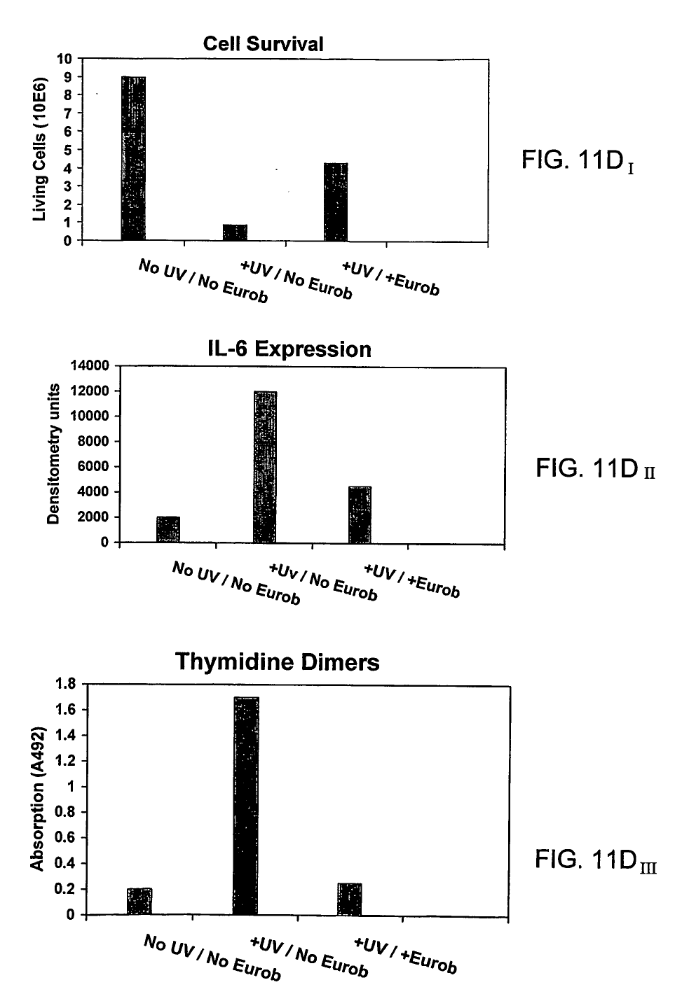

FIGS. 5A and 5B show the UVA/UVB ratio of the extracts obtained from the two cyanobacteria, Spirulina and Aphanizomenon. The figures clearly show that the UVA/UVB ratio of the two cyanobacteria extracts is 0.9-0.95. FIGS. 6A and 6B show the UVA/UVB ratio of two commercial sun-protecting lotions (used herein as control) demonstrating a lower UVA/UVB ratio of 0.7 in FIG. 6A and 0.88 in FIG. 6B. FIG. 7 shows the ultra violet spectrum of a purified compound isolated from Collema lichens. The spectrum is similar to that of the crude CAF extract (FIG. 1A), thus demonstrating that this compound is the active material responsible for the ultra violet absorbency of the CAF extract. The chemical formula of the purified compound and its complex 1D-Nuclear Magnetic Resonance spectrum are given in FIG. 8. FIG. 9 further shows the mass spectrum of the purified compound confirming its molecular formula as deduced by NMR FIG. 10 shows the effective in vitro activity against ultra violet irradiation (UVB) of the compound purified from Collema lichens (compound of formula I). The activity is demonstrated by means of survival of cultured human keratinocytes coated by the purified compound and irradiated by ultraviolet radiation and the protection in terms of pyrimidine dimer formation in cultured human keratinocytes after ultraviolet radiation. FIGS. 11A-D further show the effective in vitro activity against ultra violet irradiation (UVB) of the extracts obtained from CAF, Eriobotrya, Pecan and Cichorium. The activity is demonstrated by means of survival of cultured human keratinocytes coated by each of these extracts and irradiated by ultraviolet radiation, the protection in terms of pyrimidine dimer formation in cultured human keratinocytes after ultraviolet radiation and by measuring immunosuppression as indicated by the expression of 1-6 mRNA.

The present invention relates to extracts from natural species wherein the extracts have an ultra violet absorbency both in the UVA and UVB regions and their use in cosmetic preparations providing effective sun protection activity. The extracts of the present invention are from microorganisms such as fungus, cyanobacteria, plants, lichens or mixtures thereof. Such microorganisms, plants and lichens live in an environment exposed to strong solar radiation and have developed an effective ultra violet protection system enabling them to be exposed for long periods to sunlight that comprises ultra violet irradiation, with no apparent damage in their function. In particular the present invention concerns C₁₋₇-alcoholic extracts from the fungal part of the Collema which is termed collema associated fungus (CAF), from the cyanobacteria Aphanizomenon and Spirulina, from the plants Pecan nuts, Cichorium endivia subsp. Divaricatum—a wild plant, Eriobotrya nuts and from the lichen Xanthoria. The CAF after isolation from the Collema may easily be grown on PDA medium.

The naturally occurring material used as source for the isolation of the extracts of the present invention were grounded and the active material extracted with a C₁₋₇-alcoholic solution. The alcoholic solution may be an aqueous solution or a solution comprising a mixture of alcohol and a hydrophobic organic solvent. The C₁₋₇-alcohol used in the present invention are straight or branched C₁₋₇-alcohols. The aqueous alcoholic solution comprises H₂O:ROH in a ratio from 50:50 (%) to 5:95 (%). Particular non-limiting examples of the organic solvent used in the present invention are selected from the group consisting of alkyls, chlorinated alkyls, esters, ketones, aldehydes and aromatics. The UV absorbency of each extract is determined. The extracted compounds are very strong UV absorbers that absorb strongly in the spectral region from 220 to 425 nm. Of particular interest is the fact that the two extracts from cyanobacteria, Spirulina and Aphanizomenon display UVA/UVB ratios of 0.9 to 0.95. Such a UVA/UVB ratio, which is close to unity, reveals that these extracts are materials having effective absorbance activity in the UVA region.

In addition to the alcoholic extracts, an extract from Collema lichens was further purified to yield a compound of formula I (R and R₁-R₁₀ are hydrogen and X is NH) whose chemical structure was elucidated (see FIG. 8). The determination of the chemical structure was done using IR, UV and NMR. The IR spectrum revealed the presence of a carbonyl, primary amine and a double bond. These three functional groups are part of the mycosporine skeleton, the aminocyclohexanone ring. The existence of the primary amine group was also confirmed by ninhydrin. Various 1-Dimensional and 2-Dimensional NMR pulse sequences (DEPT, COSY, NOESY, HMBC, HSQC—data not shown) were further used to elucidate the entire structure of the pure isolated extract. From these various NMR experiments it may be deduced that the isolated compound consists of a glucoside residue bonded via pyrrolidine ring to an aminocyclohexanone ring. The pyrrolidine ring, in its meta position, has a further C₂H₄OH chain bonded to the ring via an oxygen. It should be mentioned that aminocyclohexanone moiety is part of the known elucidated structure of mycosporines, one of the naturally occurring pigments found in lichens. However, the presence of the pyrrolidine ring renders the compound a new member of the mycoscopine family.

The naturally occurring compound of formula (I) extracted form Collema lichens may easily be derivatized to yield compounds of formula (1) where R¹-R⁸ which may be the same or different selected from the group consisting of hydrogen, a C₁-C₁₀-alkyl or acyl group; R⁹ and R¹⁰ which may be the same or different are selected from the group consisting of C₁-C₁₀-alkyl, aryl, hydrogen or an acyl group; and X is NR, oxygen or sulfur, wherein R is hydrogen, alkyl or aryl. The rational of derivatization of the isolated natural compound is that the naturally occurring compound of formula I is hydrophilic, while an effective sun screen compound should be hydrophobic in order for the compound to remain on the skin despite external humidity.

It should however be understood that the use of the extracts or the purified compound or its derivatives of formula I as effective sun protection lotions, may also be by encapsulating them in appropriate encapsulating agent thus rendering their environment hydrophobic and aiding in dispersion. Alternatively they may be used together with at least one additional organic or inorganic sun protecting agent. Non limiting examples of the at least one additional sun protecting agent are derivatives of anthranilates, benzophenones, camphors, cinnamates, dibenzoylmethanes, p-aminobenzoates, salicylates, zinc oxide, titanium dioxide and mixtures thereof.

Evaluation of the sun-protecting activity of the various extracts of the present invention and of the compound of formula I in terms of preventing UVB and/or UVA induced damage as a result of irradiation was assessed both in vitro and in vivo. Such assessment was done using cell death, immunosuppression, and DNA damage as biological endpoints. In vivo determination was done by applying extracts obtained from CAF, Cichorium, Eriobotrya or pecan dissolved in olive oil on the volar forearm. Olive oil served as the control. Treated skin was totally protected from UVB induced erythema, while olive oil did not show such protection (FIG. 4).

In vitro evaluations were done using cultures of the human keratinocyte cell line, HaCaT which were irradiated with 200 mJ/cm² (cell death and immunosuppression) or 60 mJ/cm² (DNA damage) UVB delivered from a bank of four FS40 fluorescent lamps that emit wavelengths between 280 and 320 nm, with a peak at 313 nm. The cells were irradiated through a quartz plate on which solutions of the compounds were spread, and harvested immediately (DNA damage) or after 24 hours (cell death). Cell death was evaluated by vital staining, immunosuppression by IL-6 mRNA expression, and DNA damage was assayed with a polymerase chain reaction using primers for pyrimidine dimers and ELISA for staining of the cDNA product. Irradiation through naked quartz plated served as control. These tests confirm in terms of photoprotection the biological activities that were postulated based on the UV absorbing property of the extracts (FIGS. 11A-D).

### EXAMPLES

Chemical

Extraction of sun-protecting agents from various natural sources.

### Example 1

### Aphanizomenon flos aquae (Upper Klamath Lake, USA)

10 gr. of green powder (capsules comprising of Aphanizomenon Flos Aquae made by SOLGAR, IL) was transferred into a 500 ml flask connected to a condenser. 250 ml of 75% aqueous Methanol were added, and the solution was boiled for one hour. After reaching room temperature the extract was filtered through a Bichner funnel (Schleicher & Schuell 595 filter papers). The extract (intense green color) was transferred into a 500 ml separatory funnel. A mixture of 100 ml CH₂Cl₂ and H₂O was added. The layers separated, where the organic phase remains as an emulsion (green) and transferred into flask I.

The upper aqueous layer (clear and yellow) was transferred to a second separatory funnel and the extraction procedure was repeated again with the same solvent mixture in the presence of NaCl. After separation, the organic layer was transferred into flask II. The aqueous layer appeared as clear and yellow, and transferred into flask III. The organic solvent's residues from flask III were evaporated, and the water was lyophilized till dryness. 4.326 gr. of a pale yellow powder was obtained.

A sample of the powder was redissolved in methanol and checked for its UV absorbance. A UVA peak is observed at 336 nm (FIG. 1A).

### Example 2

Collema associated fungus (CAF) (one out of four fungus which exist in a Lichen known as Collema sp.) The CAF was isolated and easily grown on PDA medium.

138 gr. of fresh fungus (black) were transferred into a mortar. 100 ml of 90% aqueous methanol was added and the fungus was crushed by a pestle. The crushed material was transferred into a beaker and heated at 60° C. for 3 min. After reaching room temperature the material was filtered through a funnel (Schliecher & Schuell 593^{1/2} filter papers). The extract was transferred into a 250 ml flask and the solvents (organic and water) were moved till dryness yielding a pale yellow powder.

The powder was dissolved in 100 ml H₂O, filtered through cotton and transferred into a separatory funnel. The upper organic phase is colorless, while the lower aqueous phase appears as clear yellow. The aqueous layer was lyophilized to dryness, and 28 mg of pale yellow powder were obtained.

A sample of the powder was diluted in 70% aq. methanol and UV absorbance was measured. As a blank we used PDA medium that was dissolved in 70% aq. methanol. A UVB peak at 311 nm (FIG. 1B) was found.

### Example 3

### Pecan

100 gr of Pecan nuts (purchased on local market in Jerusalem, Ill.) were homogenized in a high-speed unit and transferred into a 500 ml flask connected to a condenser. A mixture of 100 ml of methanol and 100 ml CH₂Cl₂ were added, and solution was boiled for 1 h. After reaching to room temperature the extract was filtered through a Bichner filter. The extract (green-brown) was transferred into a 500 ml separatory funnel. A mixture of 100 ml C₂H₂ and 200 ml cold H₂O (with ice) was added. The layers were separated. The organic phase (yellow-bright) was transferred into flask. The organic layer was dried over MgSO₄. The lipid-soluble extract has UV absorbance 294 and 302 nm (FIG. 2A).

### Example 4

### Cichorium endivia subsp. Divaricatum

500 gr dried parts of Cichorium endivia subsp. Divaricatum (The wild plant Cichorium endivia L. subsp. divaricatum (Schousb.) P.D. Sell was collected at Sedot Micha, Ill.) stalks, inflorescences, and roots were separately homogenized in a high-speed unit. Homogenized roots were transferred into a 2000 ml flask connected to a condenser. A mixture of 500 ml of ethanol and 300 ml water were added, the solution was boiled for 6 hrs. After reaching to room temperature the extract was filtered through a Bichner filter. The ethanol-water extract (yellow-brown) was transferred into a 1500 ml separatory funnel. A mixture of 300 ml C₂H₂ and 300 ml cold H₂O (with ice) was added. The layers were separated. The organic phase remains as an emulsion (brown) and transferred into flask 1 (Fraction No. 1). The methanol-water layer (upper layer) was evaporated in-vacuo and the resulting oil dissolved in 50 ml dry ethanol (Fraction No 2). To Fraction No 2 was added 50 ml (×3) hexane (Fraction No 3). The layers were separated, and hexane layer was dried over MgSO₄. The three fractions gave three different absorption patterns (FIG. 2B): Fraction No. 1 has UV absorbance 294 nm. Fraction No. 2 has UV absorbance 280 nm; and Fraction No. 3 has UV absorbance 283 and 312 nm.

### Example 5

### Eriobotrya Nuts

100 gr of fresh Eriobotrya nuts (purchased on local market in Jerusalem) were homogenized in a high-speed unit and transferred into a 500 ml flask connected to a condenser. A mixture of 100 ml of methanol and 100 ml CH₂Cl₂ were added, and the solution was boiled for 1 hr. After reaching room temperature the extract was filtered through a Bichner filter. The green-brown extract was transferred into a 500 ml separatory funnel. A mixture of 100 ml C₂H₂ and 200 ml cold H₂O (with ice) was added. The layers were separated. The organic phase (green-brown) was transferred into flask. The organic layer was dried over MgSO₄. The lipid-soluble extract has a UV absorbency at 298 nm and 306 nm (FIG. 3A).

### Examples 6 & 7

Spirulina and Xantoria were extracted by similar methods and their ultra violet spectra are given in FIGS. 3B and 3C.

### Example 8

### Extraction of a Compound of Formula I (R¹-R¹⁰=H; and X=NH)

Collema (taken from sun exposed rocks near Jerusalem) were washed from sediments grounded, immersed in an H₂O/C₃OH solution (1:1). The mixture was heated to a temperature of 60° C. for 30 minutes and filtered. The biological material was subjected to another two consecutive extraction cycles with the same solvent system. The combined extracts were evaporated, immersed in dichloromethane to separate soluble lipid compounds, water added and the aqueous fraction separated. The water were lyophilized and the residue was redissolved in 90% CH₃OH/H₂O and the U.V. spectrum of the material was measured. Dry material extracted from Collema lichens showed significant U.V. absorption (FIG. 7) and was further analyzed for its structure and activity. Thin layered chromatography revealed the presence of several components. Separation of the various components and isolation of the active component having an intense U.V. absorption was done by HPLC (RP₁₈; Gradient elution of from 90% 0.05% acetic acid in water: 10% acetonitrile to 10% of 0.05% acetic acid in water: 90% acetonitrile; flow rate of 1 ml/min). The fraction eluted with RT of 14.1 min. gave the intense U.V absorbance (data not shown).

### Example 9

### Determining the Chemical Structure of the Extracted Compound of Formula I

An infra red spectrum (FTIR) of the extracted material (data not shown) revealed the presence of —OH, —NH and C═O groups (3382, 2936 and 1653 cm⁻¹, respectively). Various 1-dimensional and 2-dimensional Nuclear Magnetic Resonance experiments (DEPT, COSY, HSQC, HMBC, NOESY and TOCSY— not shown) were carried on 400 MHz and 600 MHz instruments in order to elucidate the chemical structure of the extracted compound.

The ¹H (FIG. 8) and ¹³C spectrum (not shown) were too complex for an interpretation, but they clearly demonstrated the presence of an anomeric proton and 16 carbons. Partial structure was confirmed by COSY, DEPT, HSQC, HMBC and NOESY experiments. The COSY revealed the existence of a glucoside and three other systems:

The first system consists of four protons assigned as 1′-4′, which create a pyrrolidine ring through a withdrawing group (Nitrogen).

The second system is a four protons, named as Xa, Xb, Ya, Yb having the following interactions:
Xa→Xb, Yb
Xb→Xa, Ya
Ya→Xb, Yb
Yb→Xa, Ya
   this short chain is connected to a meta position of the pyrrolidine through an oxygen atom.

The third is a five protons system assigned as C-F, which then by all other experiments was found to be a part of an aminocyclohexenone ring.

| Carbon | δ ° C. | | | | |
|---|---|---|---|---|---|
| number | (ppm) | HSQC | δ H (ppm) | DEPT | HMBC |
| 1 | 104.9 | 1g (d) | 4.45 | CH | 1′ |
| 2 | 79.1 | 1′ (d) | 4.02 | CH | 1g, 3′ |
| 3 | 78.3 | 3g | 3.37 | CH | 2g, 4g |
| 4 | 78.1 | 5g | 3.35 | CH | |
| 5 | 75.5 | 2g (t) | 3.24 | CH | 3g, 5g |
| 6 | 73.3 | C | 3.7 | CH | |
| 7 | 72.8 | 2′ | 3.84 | CH | |
| 8 | 72.7 | 4g (t) | 3.19 | CH | |
| 9 | 72.2 | 3′ | 3.86 | CH | X, Y |
| 10 | 71.9 | 4′ (d) | 3.71 | CH | D, C |
| 11 | 71.7 | D | 3.72 | CH | F_{b} |
| 12 | 65.4 | Yₐ, Y_{b} | 3.76, 3.6 | CH₂ | |
| 13 | 65.4 | Xₐ, X_{b} | 3.82, 3.63 | CH₂ | |
| 14 | 65.4 | Fₐ, F_{b} | 3.81, 3.62 | CH₂ | |
| 15 | 64.1 | E | 3.74 | CH₂ | |
| 16 | 63.6 | 6gₐ, 6g_{b} | 3.92, 3.57 | CH₂ | 4g |

Analysis of the various 1D and 2D NMR experiments give rise to a chemical structure as shown in FIG. 8. Such a compound has a chemical formula of C₁₉H₃₂N₂O₁₃ and its molecular weight is 496. Confirmation for such a chemical structure and formula was obtained from the Mass Spectrum of purified material. The mass spectrum is shown in FIG. 9A as the Time of Flight Electron Spray Ionization (TOF ESI) reveals a molecular peak of 497.026 corresponding to the MH+. FIG. 9B further shows a simulation of the proposed molecular peak at 497.198 corresponding to C₁₉H₃₂N₂O₁₃.

Biological Activity

### Example 10

### In Vivo Activity of Extracts

The in vivo biological activity was assayed by the ability to prevent UV induced erythema of human skin. Extracts were diluted in olive oil and applied to the volar forearm of a volunteer. Olive oil without extract served as control. Fifteen minutes after application of the extracts dissolved in olive oil and the olive oil to the volar forearm, 2 MED of UVB irradiation was delivered to the treated areas, and the resulting erythema was evaluated after 24 hours. Treated skin was totally protected from UV induced erythema (FIG. 4).

### Example 11

### In Vitro Activity of Extracts

Evaluation of the biological activity of the various extracts in terms of preventing UVB induced damage was assessed in vitro using cell death, immunosuppression, and DNA damage as biological endpoints: Cultures of the human keratinocyte cell line, HaCaT were irradiated with 200 mJ/cm² (cell death and immunosuppression) or 60 mJ/cm² (DNA damage) UVB delivered from a bank of four FS40 fluorescent lamps that emit wavelengths between 280 and 320 nm, with a peak at 313 nm. The cells were irradiated through a quartz plate on which solutions of the extracts were spread, and harvested immediately (DNA damage) or after 24 hours (cell death). Cell death was evaluated by vital staining, immunosuppression by IL-6 mRNA expression, and DNA damage was assayed with a polymerase chain reaction using primers for pyrimidine dimers and ELISA for staining of the cDNA product. Irradiation through naked quartz plated served as control. The results of the biological activity are summarized in Table I and shown in FIGS. 11A_{(I,II,III)}-D_{(I,II,III)} where the following may be noted.

Collema associated fungus (CAP) at a concentration of 157 μg/cm² totally prevented UVB induced erythema 11A_{(I)} and partially prevented UVB induced cell death and cyclobutane pyrimidine dimer formation 11A_{(III)}, but did not prevent IL-6 expression 11A(II).

Cichorium extracts (6 μg/cm²) totally prevented UVB induced erythema 11B_{(I)} and partially prevented UVB induced cell death IL-6 expression 11B_{(II)} and cyclobutane pyrimidine dimer formation 11B_{(III)}.

Pecan extracts totally prevented UVB induced erythema 11C(I) and partially prevented UVB induced cell death, IL-6 expression 11C_{(II)} and cyclobutane pyrimidine dimer formation 11C_{(III)}.

Eriobotrya extracts totally prevented UVB induced erythema 11D_{(I)} partially prevented UVB induced cell death and IL-6 expression 11D_{(II)} but was not effective in preventing cyclobutane pyrimidine dimer formation 11D_{(III)}.

TABLE I

| | | UV Protection | | | | |
|---|---|---|---|---|---|---|
| | | | In vitro | | | |
| | Absor- | In | Con- | | | |
| Extract | bance | vivo | centration | Survival | IL-6 | CPD³ |
| CAF¹ | 311 nm | Yes | 157 μg/cm² | 31% | 5% | 75% |
| Cichorium | 283 nm | Yes | 6 μg/cm² | 39% | 49% | 69% |
| Eriobotrya | 305 nm | Yes | Unknown³ | 41% | 65% | 91% |
| Pecan | 305 nm | Yes | Unknown | 89% | 23% | 3% |
| Aphanizomenon | 336 nm | ND² | ND | ND | ND | ND |
| Xantoria | 333 nm | ND | ND | ND | ND | ND |

¹Collema-associated fungus
²Not determined
³Dissoved in oil in undetermined concentration
⁴Cyclobutane pyrimidine dimers

### Example 12

### Determining UVA/UVB Ratio of Extracts

Determining the UVA/UVB ratio is intended to give an indication of the scope of the UV absorbence properties of a test product as part of a “broad spectrum” claim (H W Lim et al. (2001) JAAD 44:505-508).

Using UV transmittance spectrophotometry, UV radiation was directed onto the surface of a smooth quartz glass plate and the quantity of radiation transmitted through the plate was measured. UV transmission through the plate was measured at 5 nm increments throughout the UVB and UVA regions (290 nm to 400 nm) at single discrete regions of the plate to determine 100% transmission.

A roughened quartz glass plate was coated with the appropriate natural extract whose UV absorbency was measured. Coating was done in the following manner. The natural extract was dissolved in water at a concentration of 10 mg/mL and the solution was applied in a series of small dots to the roughened quartz glass plate using a micro-pipette and then spread evenly using a gloved finger to achieve a typical uniform application rate of 0.75 mg/cm² of product. Care was taken to ensure the formation of a uniform film. The coating was allowed to dry for ca. 10 minutes. Radiation from a U.V. source was directed onto the surface of the coated plate and the quantity of radiation transmitted through the coated plate was measured. UV transmission was measured at 5 nm increments from 290 nm to 400 nm at 6 discrete regions of the plate. This procedure was carried out 3 times for each of the extracts whose ultra violet absorbency was measured. Two internal control containing commercial sun-protecting products agents were tested prior, and after the irradiation extract, for validation purposes. The two internal control products are hereinafter termed “nivea 3 star” (SPF 12) and “boots soltan 4 star” (SPF 4). The transmission measurements obtained before and after the substrate coating at discrete wavelength intervals from 290 nm to 400 nm was used to give an indication of the protection potential of the product throughout the UVA and UVB regions.

The “critical wavelength” and the UVA protection categories were then determined as shown in the Table below (according to the Boots the Chemists Ltd UVA symbol system based upon an in vitro technique previously described by B. L. Diffey and J. Robson (J. Soc. Cosmet. Chem, 40, 127-133, May/June 1989)). A “Critical wavelength” value equal or greater than 370 nm is required for broad-spectrum protection claim. The “Boots” Star method protection categories are summarised in Table II.

TABLE II

| Mean UVA/UVB Protection Ratio | UVA Protection Category |
|---|---|
| 0-<0.2 | Too low for UVA claim |
| 0.2-<0.4 | Moderate (*) |
| 0.4-<0.6 | Good (**) |
| 0.6-<0.8 | Superior (***) |
| 0.8+ | Maximum (****) |

The mean and standard deviation UVA/UVB protection ratios based on three measurements done for each substrate, critical wavelength and percentage UVA block calculated for the two extracts and the control compounds are given in Table III.

TABLE III

| | | | UVA protection |
|---|---|---|---|
| | Mean critical | Mean | category |
| Substrate | wavelength | UVA/UVB ratio | (Table II) |
| Nivea 3 star | 377 | 0.64 ± 0.01 | Superior (***) |
| Boots soltan 4 star | 380 | 0.88 ± 0.01 | Maximum (****) |
| Spirulina | 388 | 0.92 ± 0.01 | Maximum (****) |
| Aphanizomenon | 388 | 0.95 ± 0.01 | Maximum (****) |
| flos aqua | | | |
| Nivea 3 star | 375 | 0.68 ± 0.01 | Superior (***) |
| Boots soltan 4 star | 380 | 0.89 ± 0.01 | Maximum (****) |

### Example 13

### In Vitro Activity of the Compound of Formula I (Purified From Collema lichens)

A compound of formula I at a concentration between 0.5 to 3 mg/cm² was spread on a quartz plate, which covered the dish, containing the cultured cells. Four FS-40 fluorescent lights radiating at wavelengths between 280 and 320 nanometers served as the source of UVB. These lamps were placed at a distance of 20 cm from the quartz plate. Under the same conditions of irradiation a cultured cell preparation was irradiated with no protection, i.e. naked quartz plate.

A: Cells Survival.

FIG. 10A shows the cell survival after 200 mJ/cm² UVB irradiation. UVB protection was offered in a dose-dependent manner, since 74% of the cells survived when cells were protected with 3 mg/cm² of the compound whereas only 43% survived with 0.16 mg/cm² of the compound. Only 20% of the cells survived when irradiated through the naked quartz plate.

B: DNA Damage

FIG. 10B shows generation of pyrimidine dimers after 60 mJ/cm² UVB irradiation through quartz plates covered with 6 mg/cm² of the formula (I) compound, commercial sunscreen or naked quartz plate. Non-irradiated cells showed a low background of pyrimidine dimers. Plates covered with the formula (I) compound offered around 80% protection compared to cells irradiated through the naked quartz plate.

Although the invention has been described in conjunction with specific embodiments, it is evident that many alternatives and variations will be apparent to those skilled in the art in light of the foregoing description. Accordingly, the invention is intended to embrace all of the alternatives and variations that fall within the spirit and scope of the appended claims.

1. A natural extract from fungus, cyanobactria, plants, lichens or a mixture thereof having an ultra violet absorbency in the range of 220 nm to 425 nm wherein said extract is obtained by contacting said fungus, cyanobacteria, plant, lichen or a mixture thereof with an C₁₋₇-alcoholic solution.

2. A natural extract according to claim 1, wherein the alcoholic solution is an aqueous alcoholic solution comprising H₂O:ROH in a ratio of from 50:50(%) to 5:95(%).

3. A natural extract according to claim 1, wherein the alcoholic solution comprises a hydrophobic organic solvent selected from the group consisting of alkyls, chlorinated alkyls, esters, ketones, aldehydes or aromatics.

4. A natural extract according to claim 1, wherein the fungus is Collema associated fungus, the plant is chosen from the group comprising of Pecan nut, Cichorium endivia, Eeriobotrya nut or mixtures thereof, the lichen is Xanthoria and the cyanobacteria is chosen from the group consisting of Spirulina or Aphanizomenon or mixture thereof.

5. A compound of formula (I) 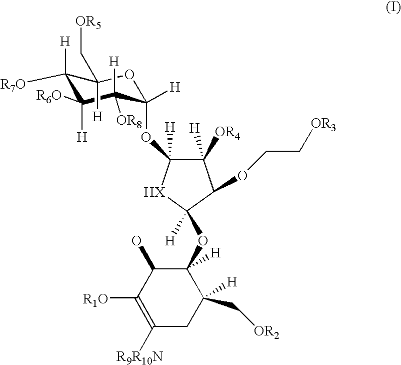
wherein R¹-R⁸ which may be the same or different are selected from the group comprising hydrogen, a C₁-C₁₀-alkyl or acyl group; R⁹ and R¹⁰ which may be the same or different are selected from the group comprising of C₁-C₁₀-alkyl, aryl, hydrogen or an acyl group; and X is NR, oxygen or sulfur, wherein R is hydrogen, alkyl or aryl.

6. A compound according to claim 1 wherein R¹-R⁸ are hydrogen, R⁹ and R¹⁰, which may be the same or different are selected from the group comprising of C₁-C₅-alkyl, aryl, hydrogen or an acyl group and X is NH.

7. A compound according to claim 1 wherein R, R₁-R₁₀ are hydrogen and X is NH.

8. A cosmetic formulation for providing protection from ultra violet irradiation comprising an effective amount of an extract of claim 1.

9. A cosmetic formulation for providing protection from ultra violet irradiation comprising an effective amount of a compound of formula (I) of claim 5 together with at least one cosmetically acceptable excipient.

10. A cosmetic formulation according to claim 8 further comprising an additional sun-protecting agent.

11. A cosmetic formulation according to claim 9 further comprising an additional sun-protecting agent.

12-14. (canceled)

15. A cosmetic formulation according to claim 11 wherein said additional sun-protecting agent protects against UVA or UVB.

16. A cosmetic formulation according to claim 10, wherein said additional sun-protecting agent protects against UVA or UVB.

17. A cosmetic formulation according to claim 10, wherein said additional sun-protecting agent is selected from the group consisting of derivatives of anthranilates, benzophenones, camphors, cinnamates, dibenzoylmethanes, p-aminobenzoates, salicylates, zinc oxide, titanium dioxide and mixtures thereof.

18. A cosmetic formulation according to claim 11, wherein said additional sun-protecting agent is selected from the group consisting of derivatives of anthranilates, benzophenones, camphors, cinnamates, dibenzoylmethanes, p-aminobenzoates, salicylates, zinc oxide, titanium dioxide and mixtures thereof.

19. In a method of protecting the skin against adverse effects of UV, comprising applying to the skin a sun-protecting composition, the improvement wherein said composition comprises the compound of claim 5.
